(19) United States
(12) Patent Application Publication
(10) Pub. No.: US 2024/0099713 A1
(43) Pub. Date: Mar. 28, 2024

(54) SURGICAL SOFT TISSUE SUTURING DEVICE

(71) Applicant: INNO4SPINE AG, Stans (CH)

(72) Inventors: Heiko KOLLER, Kufstein (AT); Tamás Fülöp FEKETE, Zürich (CH); Daniel HASCHTMANN, Kusnacht (CH); Dezsö János JESZENSZKY, Kusnacht (CH); Tom OVERES, Attiswil (CH)

(21) Appl. No.: 18/274,688

(22) PCT Filed: Nov. 30, 2021
(86) PCT No.: PCT/IB2021/061124

§ 371 Date: Jul. 27, 2023

(30) Foreign Application Priority Data
Feb. 2, 2021 (CH) 00099/21

(51) Int. Cl. A61B 17/06; A61B 17/04

(52) U.S. Cl. CPC A61B 17/06166; A61B 17/0467; A61B 17/06066; A61B 2017/061; A61B 2017/06176

(57) ABSTRACT

According to one aspect, a surgical suturing device (10) is proposed for closing an incision or two layers of a target soft tissue (70). The suturing device (10) comprises: a shuttle driving mechanism (14) for driving a shuttle (36) of a suture holder assembly (30); a soft tissue window (22) for receiving at least a portion of the target tissue (70); an ejection window (23) sized and shaped to allow a suture holder assembly (30) to be ejected out of the suturing device (10) through the ejection window (23); and a chamber (26) for receiving a set of suture holder assemblies (30). The suturing device can be used to create suture loops through the target tissue.

### TECHNICAL FIELD OF THE INVENTION

The present invention relates to a surgical soft tissue suturing device. The suturing device may be used after a surgical intervention to close two portions of a target soft tissue arranged at opposite sides of an incision. The suturing device may be used in combination with bio-resorbable or permanent suture material. The proposed surgical suturing device provides a surgeon with a tool to close incisions in a fast and secure manner with a high resistance to forces promoting wound dehiscence. The invention also relates to a surgical suturing tool assembly and to a surgical suture holder assembly comprising a suture and an attached shuttle for passing the suture.

### BACKGROUND OF THE INVENTION

After an invasive surgical intervention, often a longer incision needs to be closed. Most commonly sutures are used to close the muscle and the fascial layer, and sutures or metal staples are used to close the skin. Depending on the type of tissue layer to be closed, bioresorbable or permanent materials may be preferred. Longer skin incisions are frequently closed with staples. These staples are made of metal and clamp the skin together. These metal staples are removed after healing. These staples, which are placed in the outer tissue layer of the body, can generally be easily removed. The staples are bent backwards using a special tool, and then pulled out.

Metal staples are rarely used for deeper tissue layers, for instance because the removal would be too difficult, and a new incision would be needed. Furthermore, the staples are not configured to stay long in the body. For example, the staples can break due to stress. Therefore, in these body parts, most often sutures are used. Sutures are strong and can be chosen out of resorbable and non-resorbable materials. The disadvantage of sutures is that they are very time consuming to place.

Thus, in view of the above, there is a need for an improved tissue suturing device or tool that provides a surgeon with a fast and secure application or deployment technique to close longer and short incisions in the high-stress or deeper tissue layers, as well as in superficial skin layers. In other words, there is a need for a suturing device loaded with multiple suture strands that can be applied to a target incision and that provides increased security, reproducibility, and reduced surgery time.

### SUMMARY OF THE INVENTION

It is an object of the present invention to overcome at least some of the problems associated with the closure of incisions in deeper soft tissue layers, such as the myofascial tissue layers, as well as superficial skin tissue layers. There is a need for a suturing device that provides the ability of quickly placing suture loops that are made of a bioresorbable material or alternatively of a non-resorbable material. The suture loops should be able to withstand higher stresses and safely close the target tissue.

According to a first aspect of the invention, there is provided a surgical suturing device as recited in claim 1.

According to a second aspect of the invention, there is provided a surgical suturing tool assembly for closing an incision or two layers of a target soft tissue, the suturing tool assembly comprising the suturing device according to the first aspect, and a set of suture holder assemblies.

The suturing tool assembly comprises a stack of suture holder assemblies or clips with suture loops, which can be passed through the target tissue. After a first suture holder assembly has been used, a second suture holder assembly is made available for the next suture passing. Furthermore, the suture is sized and shaped in such a manner that the suture passing automatically creates a suture loop through the target soft tissue. The suturing device and/or the suturing tool assembly is/are (a) single-use instrument(s). Thus, they may be disposable instruments. Such an instrument would typically be delivered in a sterile packaging, and it may be thrown away after use.

According to a third aspect of the invention, there is provided a suture holder assembly as recited in claim 23.

Other aspects of the invention are recited in the dependent claims attached hereto.

### BRIEF DESCRIPTION OF THE DRAWINGS

Other features and advantages of the invention will become apparent from the following description of non-limiting example embodiments, with reference to the appended drawings, in which:

FIGS. 1A and 1B depict an example suturing tool assembly according to an embodiment of the present invention;

FIG. 1C depicts a sutured target tissue that has been stitched up by using the suturing tool assembly of FIGS. 1A and 1B;

FIGS. 2A to 2D depict a suture holder assembly of the suturing tool assembly of FIGS. 1A and 1B;

FIGS. 3A and 3B depict a suturing device of the suturing tool assembly of FIGS. 1A and 1B;

FIGS. 4A to 4C depict examples of a suture strand;

FIGS. 5A to 5J depict the operation of the suturing tool assembly of FIGS. 1A and 1B; and

FIG. 6 depicts an alternative design of a shuttle and a shuttle driver.

### DETAILED DESCRIPTION OF THE INVENTION

An embodiment of the present invention will now be described in detail with reference to the attached figures. The embodiment is described in the context of a suturing device and two portions of a target soft tissue arranged at the opposite sides of an incision, which is to be closed after a surgical intervention. Although the invention is specifically described in this context, the teachings of the invention are not limited to this environment. When the words first and second are used to refer to different elements, it is to be understood that this does not necessarily imply or mean that the first and second elements are somehow structurally substantially different elements or that their dimensions are substantially different unless implicitly or explicitly stated. Moreover, the word “end” is understood not only to mean the actual end point of an element but to also cover an end region in the proximity of the actual end point of the respective element. Identical or corresponding functional and structural elements which appear in the different drawings are assigned the same reference numerals.

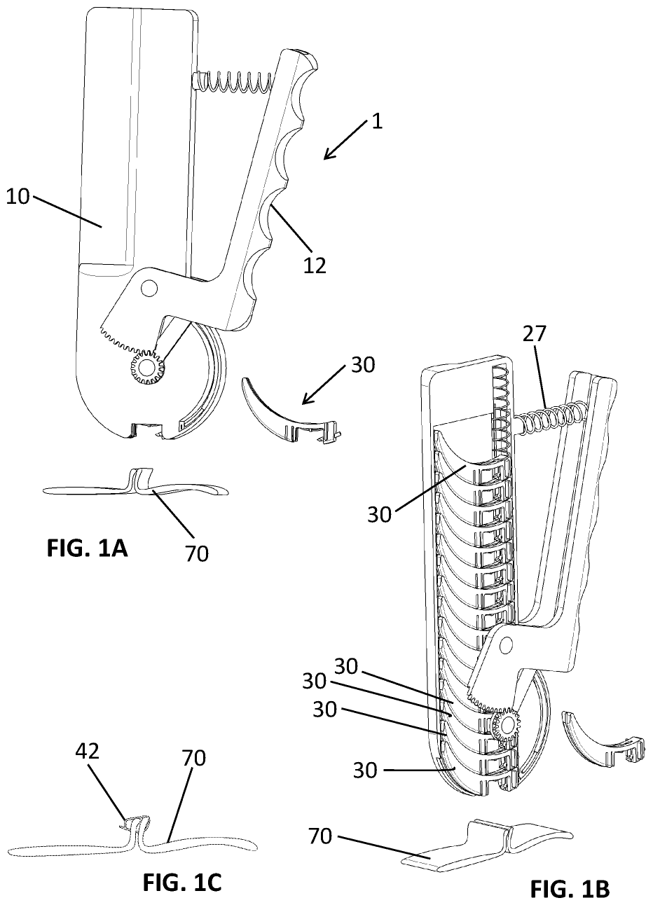

FIGS. 1A and 1B show the suturing tool assembly 1 in a perspective view and in a cross-sectional view, respectively. The suturing tool assembly 1 comprises a suturing apparatus or device 10, which is configured to accommodate or receive a set or stack of suture holder assemblies 30. The suture holder assemblies each comprises e.g. a suture strand 31, which is to be passed through a target soft tissue 70. FIG. 1C shows an example suture loop 42 or suture stitch passed through the target soft tissue 70.

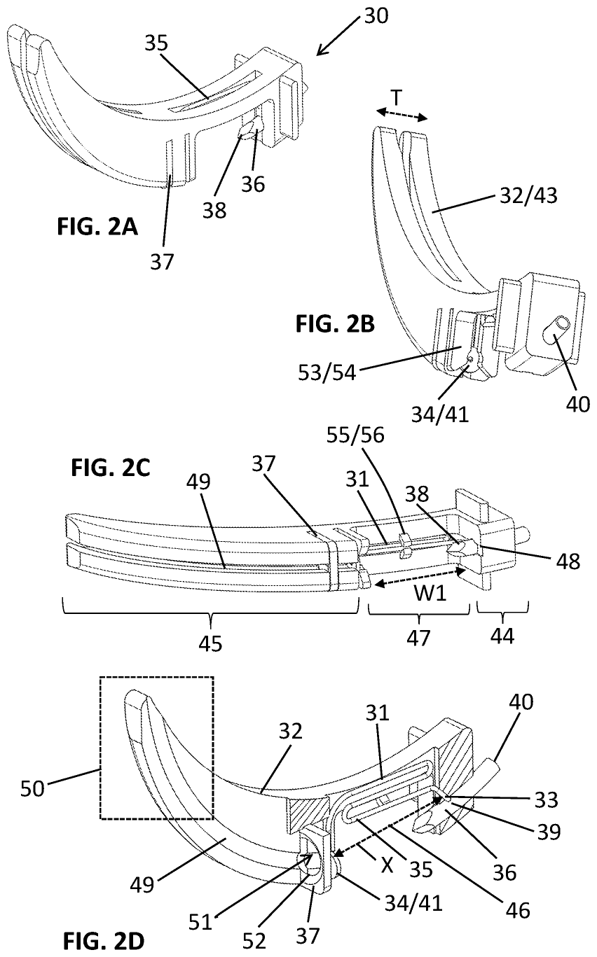

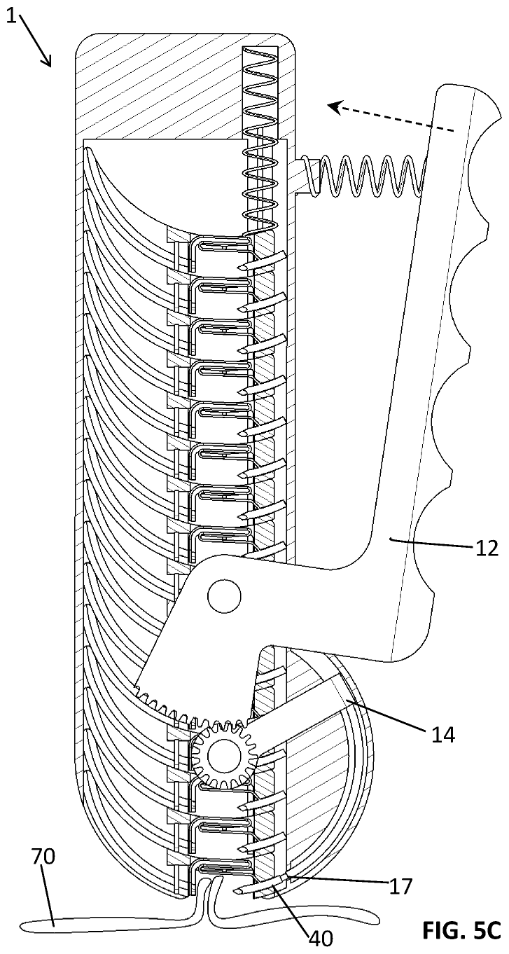

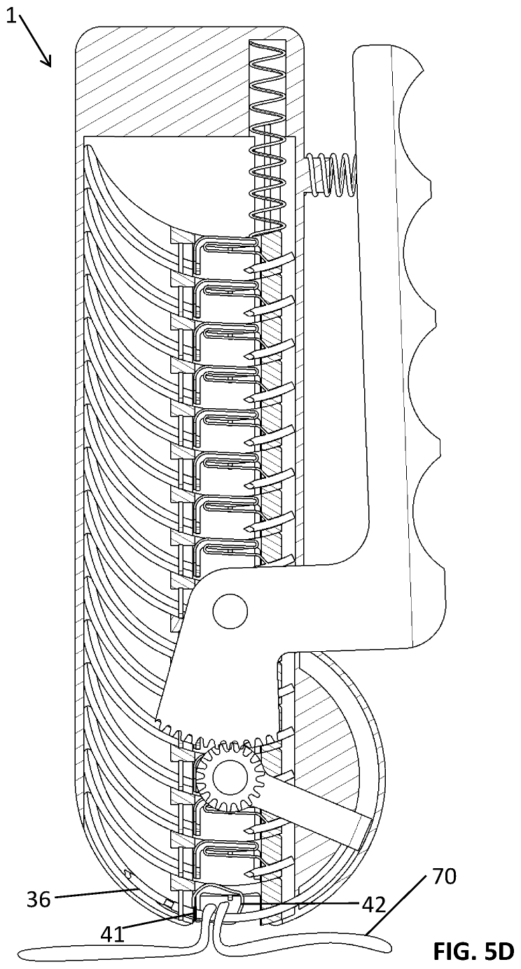

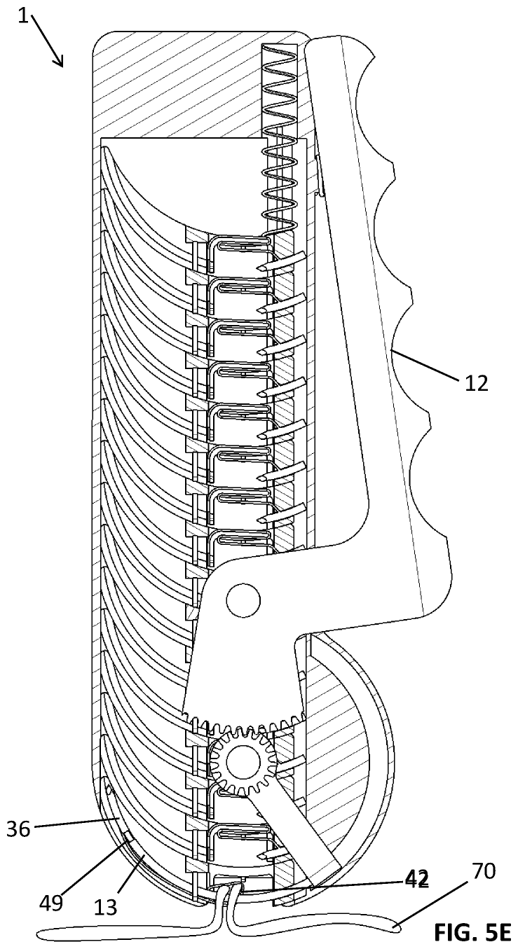

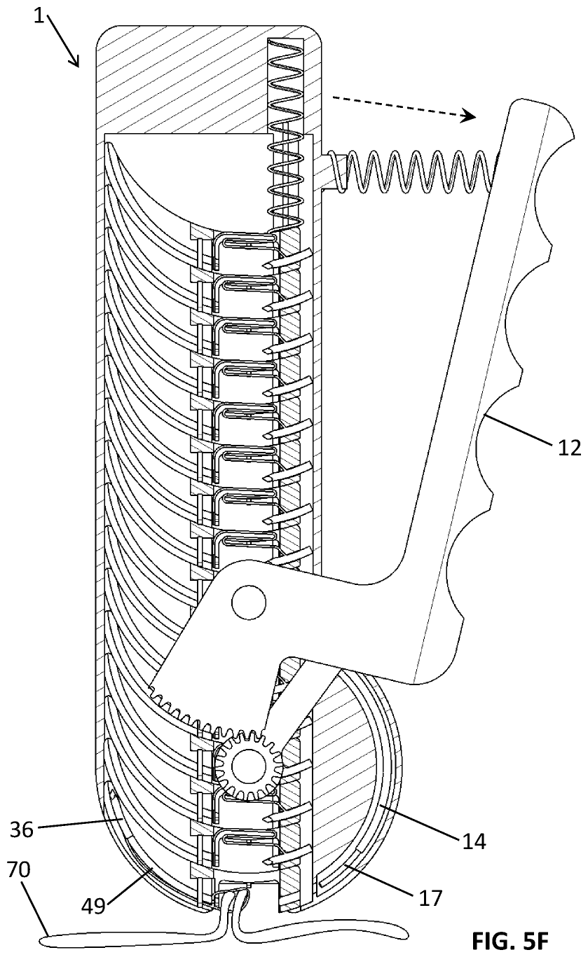

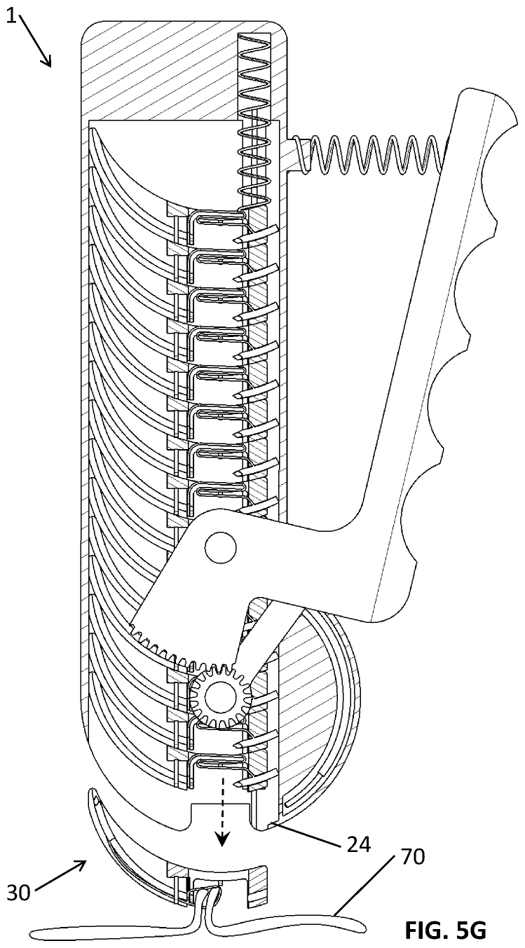

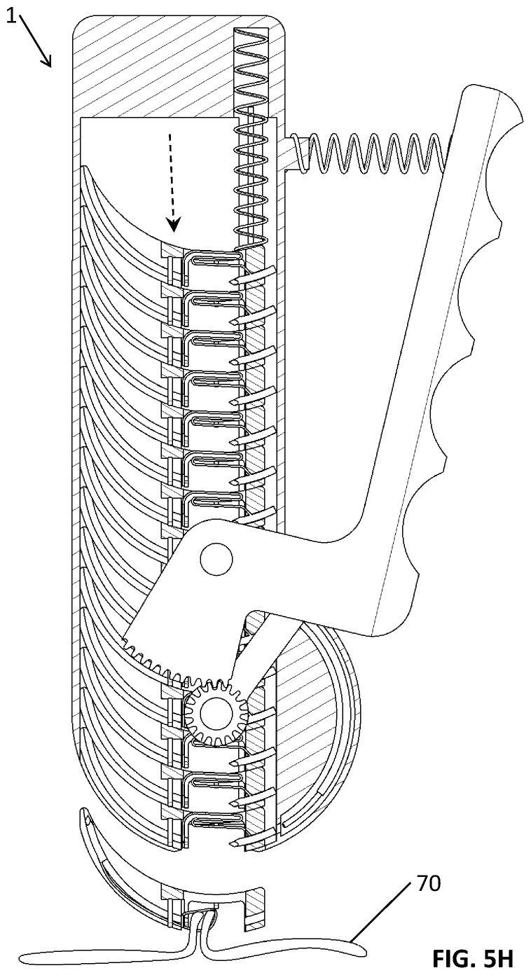

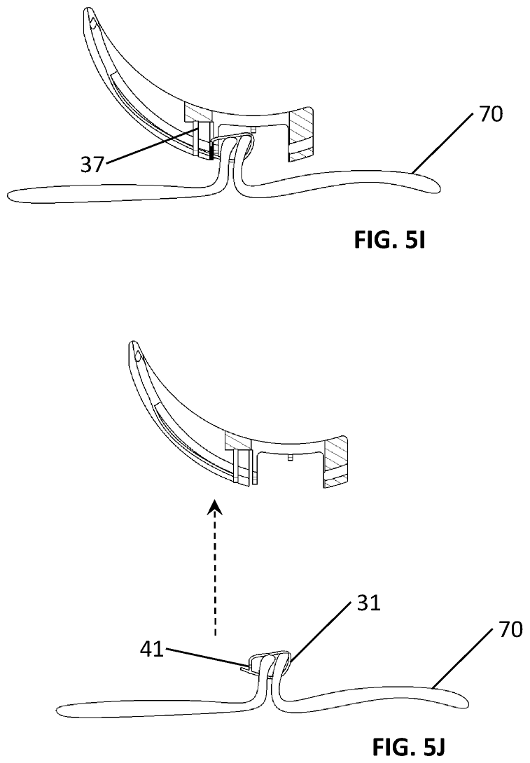

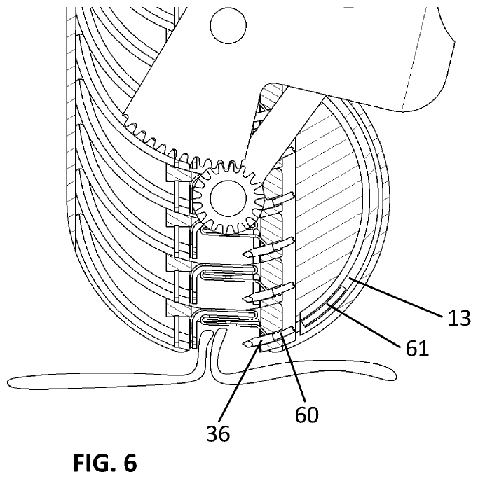

FIGS. 2A to 2D show the suture holder assembly 30 in greater detail in perspective and cross-sectional views. The suture holder assembly 30 is shown in a first or start state. As explained later in connection with FIGS. 5A to 5J, the suturing tool assembly 1, and therefore also the suturing device 10 and the suture holder assembly, defines different states, namely a first or start state, a second or engaging state, a third or transferring state, a fourth or tensioning state, and a fifth or released or end state.

The suture holder assembly 30 comprises a holder 32, a suture strand 31 having a first suture strand end 33, a second suture strand end 34 and a suture central portion 35 therebetween, a shuttle 36 and a blade 37. The shuttle, which in this example is configured as an elongated tubular element, has a needle end 38, a suture attachment portion 39 (in this case in the central or middle region or portion of the shuttle) and a driving end 40. The first suture strand end 33 is attached to the suture attachment portion. The second suture strand end is attached or connected to a suture retention element or means 41. The suture retention element 41 comprises an opening or hole, which may be a central opening, to allow the shuttle 36 to pass through it (while still being connected to the second suture strand end 34) and thus to receive the suture strand 31 and so to form a suture loop 42. As described in greater detail later, a fit, such as a form fit, a friction fit, a hooking, or other retentive fit between the suture strand (and more specifically its second suture strand end) and the suture retention element will inhibit an unravelling or releasing of the suture loop 42. As the suture retention element 41 and the suture 31 remain attached to the target tissue once the suturing operation has been completed, they can be understood to form an element similar in function to a cable tie.

In this example the holder 32 has a crescent-shaped or C-shaped holder body 43 of an average thickness “T” (measured orthogonally to the holder body's longitudinal axis). The holder body 43 comprises a first holder end or side 44 and a second holder end or side 45, a soft tissue clearance 46 between the first and second ends. The soft tissue clearance 46 is a clearance or gap, in which upon operation of the suturing tool assembly 1, the target soft tissue 70 is being arranged. As explained in greater detail later, upon operation of the suturing tool assembly 1, the shuttle 36 with an attached suture strand 31 is passed across or through this soft tissue clearance 46, and thereby forced through the soft tissue 70. The soft tissue clearance 46 is located in a central portion or section 47 of the holder 32. As depicted in the cross-sectional view of FIG. 2D, the holder 32 comprises a shuttle start fixture 48 arranged at the first holder end 44 and a shuttle receiving channel 49 arranged at the second holder end 45. The shuttle start fixture 48 is in this example configured as a hole or opening, in this case (entirely) through the first holder end 44.

In this example the shuttle receiving channel 49 has a curved shaped, more specifically a curved shape of a substantially constant radius. The shuttle receiving channel 49 comprises a shuttle end fixture 50. As explained in greater detail later, the shuttle end fixture 50 is configured to hold the shuttle 36 after it has passed through the soft tissue 70 and thus its purpose is to inhibit the shuttle 36 from inadvertently travelling back towards the soft tissue clearance 46. In this example, the end fixture is a narrowed channel section, i.e. a section with a smaller channel cross section than the cross section of the remainder of the shuttle receiving channel 49. The holding effect of the fixtures can be generated by friction, form fit, etc. In this example, the holding force is a friction-based holding force.

The holder 32 further comprises a blade 37 at the second holder end 45. The blade 37 intersects the shuttle receiving channel 49. The blade 37 comprises an opening or passage 51, which overlaps the shuttle receiving channel 49. The passage 51 is sized and shaped in such a manner that the shuttle 36 can pass through the passage 51 upon operation of the suturing tool assembly 1. The blade 37 comprises a cutting edge 52 which is directed towards the shuttle receiving channel 49 (or its centre). The cutting edge 52 has the purpose of cutting off a remaining piece of the suture strand 31, after a suture stitch or suture loop 42 has been placed, as depicted in FIGS. 5A to 5J.

At the second holder end 45, adjacent to the soft tissue clearance 46, the holder body comprises a suture retention means or fixture 53 or a suture end fixture 54. The suture retention fixture 53 is configured to hold the second suture strand end 34 in such a manner that it will receive the suture strand 31 as soon as the shuttle 36 has passed through the suture retention fixture 53. In this example, the suture retention fixture 53 intersects the shuttle receiving channel 49. Therefore, the second suture strand end 34 overlaps the shuttle receiving channel 49. In this example, the suture retention fixture 53 holds the suture retention element 41 by means of a friction fit.

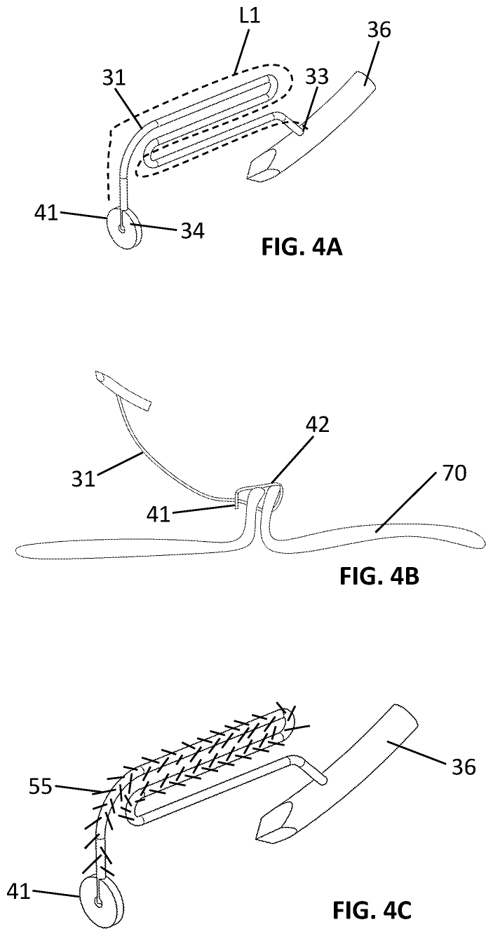

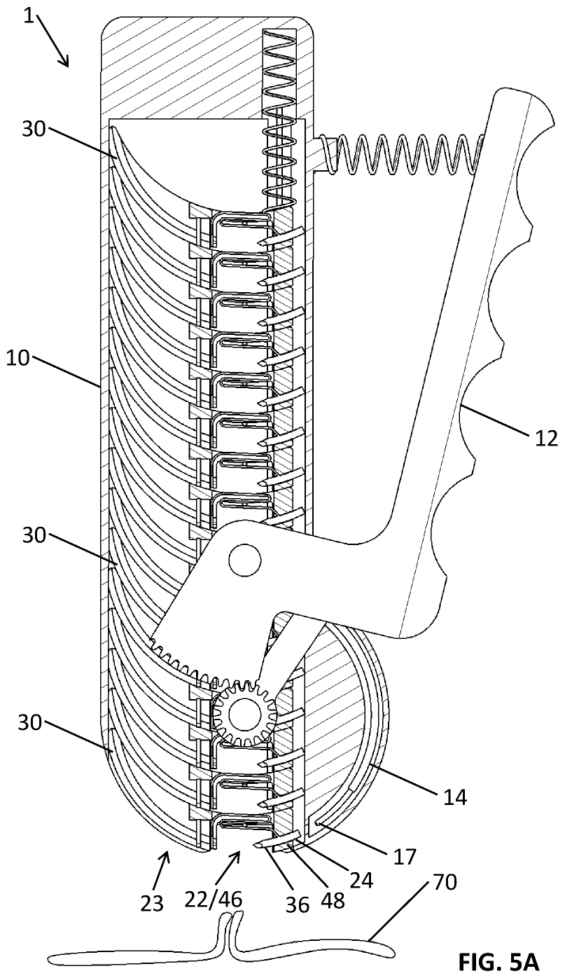

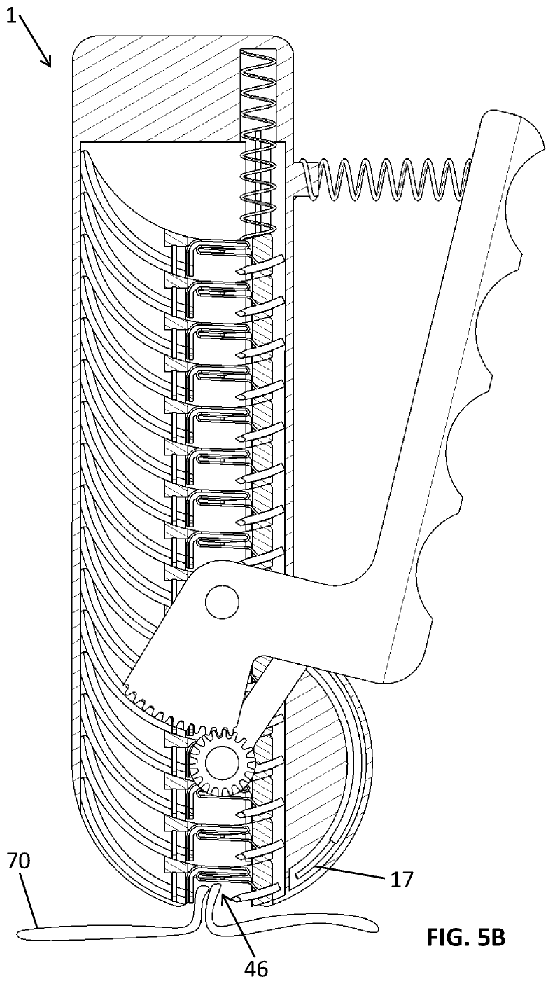

As depicted in greater detail in FIG. 4A, the suture central portion 35 is arranged between the first suture strand end 33 and the second suture strand end 34 and has a first length “L1”. This first length “L1” should have a sufficient length to allow a suture loop 42 to be formed through the target soft tissue layers, and it should further have a length which allows this loop to be adequately tensioned. As shown in FIG. 2C, the soft tissue clearance 46 has a first width “W1”. In this example, the first width “W1” is shorter than the first length “L1”. In the first state of FIGS. 2A to 2D, the first suture strand end 33 is attached to the shuttle adjacent to the soft tissue clearance 46 and the second suture strand end 34 is arranged adjacent to the suture retention fixture 53. Both suture strand ends 33, 34 are spaced a distance “X” apart, which is shorter than the first length “L1”. To prevent the suture central portion 35 from being arranged loosely in the soft tissue clearance 46, the soft tissue clearance 46 comprises a suture holding means 55 or suture clip 56. The suture clip 56 is configured to hold the suture in an organised manner and to automatically release the suture when sufficient tension is applied.

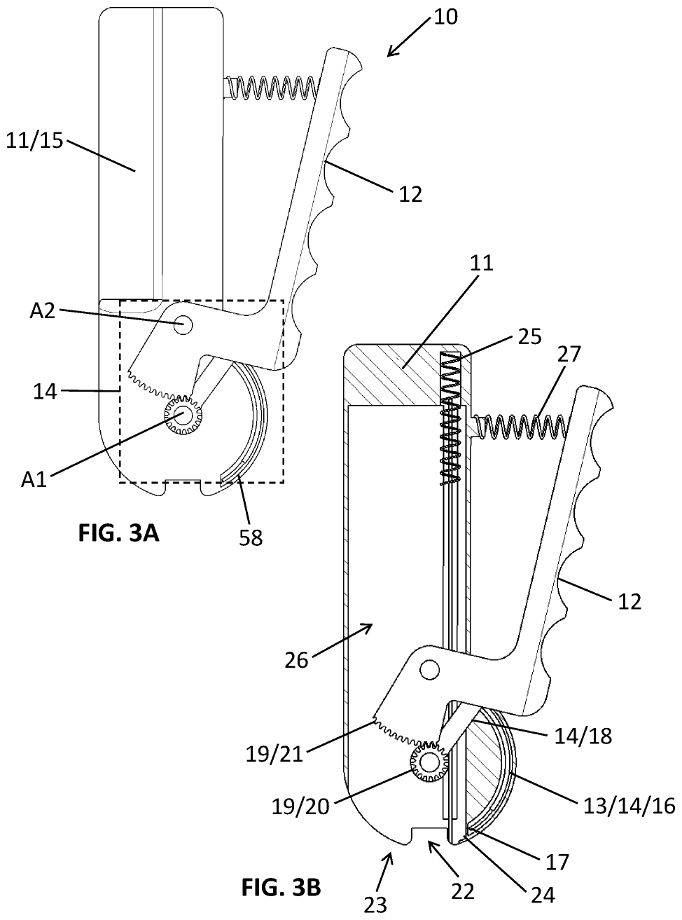

FIGS. 3A and 3B show the suturing device 10 in greater detail in a side view and in a cross-sectional view, respectively. The suturing device comprises a housing 11, a lever 12 or an activation mechanism, and a shuttle driving mechanism 14 comprising a shuttle driver 13. The housing 11 forms a handle 15 to which the lever 12 is attached or coupled. The lever 12 is configured to be operated by the fingers of the user when holding the handle 15, or vice versa. The shuttle driver 13 is configured as a thin, curved needle 16. In the present text, the word needle is to be understood as an elongated slender piece, but not necessarily having a pointy or sharp tip. Furthermore, the needle 16 may additionally be rigid or substantially rigid to facilitate driving of the shuttle as explained later. The shuttle driver 13 is configured to engage with the shuttle 36, and it forms a temporary or releasable unity when passing the shuttle 36 through the target soft tissue 70, and it allows the shuttle 36 to be released when an end position has been reached. For this purpose, the shuttle driver 13 comprises a shuttle engaging tip 17. In this example, the shuttle engaging tip 17 has a first cross-sectional diameter, and a main body portion of the needle has a second cross-sectional diameter, wherein the second diameter is greater than the first diameter.

In this example, the needle 16 is movably coupled to the housing 11 and can travel along a defined path. For this purpose, the suturing device 10 may comprise a groove or device channel 58 such that the needle 16 can be received in the groove or device channel and travel therein during the operation of the suturing device 10. More specifically, the needle 16 is rotatably coupled to the housing by an arm 18, which can also be understood to be part of the shuttle driving mechanism 14, and can rotate about a first axis A1 and therefore travels along a substantially semi-circular path in the present example. Therefore, in this example, the needle path has a substantially constant radius. The needle 16 is directly or indirectly actuated by the lever 12. In this example, the lever 12 is directly coupled to the shuttle driving mechanism 14 and more specifically to the arm 18 by a meshed cogwheel arrangement comprising at least two cogwheel sections, namely a first cogwheel or tooth section 20 and a second cogwheel or tooth section 21. In this example, the first cogwheel section 20 is in unity with the shuttle driving mechanism 14 (i.e. the shuttle driving mechanism can be understood to comprise the first cogwheel section), and the second cogwheel section 21 is in unity with the lever 12 (i.e. the lever can be understood to comprise the second cogwheel section). In other words, the shuttle driving mechanism 14 comprises at one of its ends the first cogwheel section 20, which in this example is configured as an entire cogwheel, while the lever 12 comprises at one of its ends the second cogwheel section 21. The lever 12 defines a second axis A2, which forms a pivot axis for the lever 12. In this example, the first and second axes are parallel or substantially parallel to each other. The second cogwheel section 21 has a greater diameter than the first cogwheel section 20, and therefore a specific angular rotation of the lever 12 will initiate a greater angular rotation of the shuttle driving mechanism 14.

The housing 11 further comprises a soft tissue window 22 which overlaps the soft tissue clearance 46 and an ejection window 23 which is sized an shaped to let the suture holder assembly 30 pass through. In this example, the soft tissue window is configured as an opening or recess, for example at the bottom of the suturing device. The ejection window is also configured in this example as an opening at the bottom of the suturing device. As shown for example in FIG. 1B, the suturing device 10 can hold a stack of suture holder assemblies 30 and therefore comprises an inner chamber 26. At the upper end of the stack, a feed mechanism 25, which in this example comprises a first biasing element or spring, is provided. The feed mechanism 25 is configured to feed or translate the next suture holder assembly towards the ejection window 23, after a given suture assembly 30 has been ejected, as described in greater detail later. Moreover, the suturing device comprises a stop element or seat 24 arranged adjacent to the ejection window. More specifically, the chamber 26 is delimited by a set of wall sections, and at least one of the wall sections comprises a stop element configured to engage against the shuttle 36 of the suture holder assembly 30 to prevent inadvertent ejection of the suture holder assembly out of the chamber. Thus, the purpose of the stop element is to inhibit any unwanted ejection of the suture holder assembly 30, prior to the intended passing of the suture strand 31 with the attached shuttle 36. In other words, it inhibits ejection of the suture holder assembly at least in the first and second states of the suturing tool assembly. There is also provided a second biasing element or a second spring 27 which is arranged to urge the lever 12.

FIGS. 4A to 4C show in more detail an example suture strand 31 without the holder 32. FIG. 4A depicts the suture strand 31, the suture retention element 41 and the shuttle 36. FIG. 4B shows a suture loop 42 through the target soft tissue 70. FIG. 4C shows a possible fit, such as a form fit, a friction fit, a hooking, or other retentive fit between the suture strand 31 and the suture retention element 41, which will inhibit an unravelling or 10 releasing of the suture loop 42. FIG. 4C further shows an example suture strand with barbs 55. The barbs form sharp projections extending from the suture strand, which are angled away from the suture strand towards the second end, so as to make extraction difficult, when the barbs have passed the suture retention element 41. Hence, the suture loop cannot unravel.

FIGS. 5A to 5J show the operational principle of the suturing tool assembly 1. In FIGS. 5A and 5B the suturing tool assembly 1 is shown in a first or unfired state. FIG. 5C shows a second or engaging state. FIG. 5D shows a third or transferring state. FIG. 5E shows a fourth or suture strand tensioning state. FIG. 5F shows a fifth or released state. FIG. 5G shows the situation where the suture holder assembly 30 has been ejected from the suturing device 10. FIG. 5H illustrates the automatic reloading process. FIGS. 5I and 5J show how the suture loop 42 can be released or separated from the holder 32.

FIGS. 5A and 5B show the first state of the suture holder assembly 30 (or the suturing tool assembly 1). In this state, the shuttle 36 is arranged in the shuttle start fixture 48, and the needle end 38 is directed towards the soft tissue clearance 46. In other words, in this state, the respective shuttle 36 is arranged at the first holder end 44 and disengaged from the shuttle driving mechanism 14. The first suture strand end 33 is attached to the shuttle 36, and the suture central portion 35 is arranged across the soft tissue clearance 46 in an organised manner while being held by the suture clip 56. The suture retention element 41 is arranged or sandwiched between the suture retention fixture 53 and the holder body 43, and it overlaps the shuttle receiving channel 49. In this example, the driving end 40 of the shuttle 36 protrudes from the holder 32, and is engaged with or pressed against the stop seat 24 of the housing 11. Therefore, any unwanted release of the suture holder assembly 30 from the housing 11 is inhibited. In FIG. 5B, the target soft tissue is at least partially received in the soft tissue clearance 46.

FIG. 5C shows the second or engaging state. By activating or pressing the lever 12, the shuttle driving mechanism 14 is made to rotate about the first axis A1. The shuttle engaging tip 17 becomes engaged with, or more specifically is inserted into the shuttle driving end 40. For this purpose, the shuttle 36 may comprise a shuttle channel longitudinally through at least a portion of the shuttle 36 for receiving the tip 17. Consequently, the shuttle 36 is made to move congruently with the needle 16 that is displaced or pushed by the arm 18. FIG. 5D depicts how the shuttle 36 has been forced through the soft tissue 70 and further pressed through the suture retention element 41. For this purpose, the suture retention element may be sufficiently elastic or flexible and/or it may comprise a compliant structure (such as a slot) to let through the shuttle. A suture loop 42 passing around or over and through the soft tissue 70 is thus formed.

FIG. 5E depicts the tensioning state. The shuttle 36 has been advanced forward in the shuttle receiving channel 49 and has reached the shuttle end fixture 50. The suture strand 31 and therefore the suture loop 42 is being tensioned. The shuttle has now reached its end position. At the same time the lever 12 can no longer be advanced any further as its maximum pressing degree has been reached in this case. FIG. 5F depicts the release of the lever 12 by the operator. The shuttle driving mechanism 14 is retracted, i.e. it is moved back to its initial position. Upon retraction, the shuttle 36 disengages form the shuttle engaging tip 17 and stays in the end position in the shuttle receiving channel 49. Upon disengaging, and fully retracting, the suture holder assembly 30 is fully released from the shuttle driving mechanism 14. FIG. 5G depicts how the suture holder assembly 30 has been ejected from the suturing device 10. FIG. 5H depicts how the feed mechanism 25 has fed the next suture holder assembly 30 towards the ejection window 23. FIGS. 5I and 5J illustrate the situation where by picking up the holder 32, the suture strand 31 is tensioned against or over the blade 37 and the suture strand 31 is cut off next to the suture retention means 41.

FIG. 6 depicts an alternative design of the shuttle 36 and the shuttle driver 13. In this example the shuttle comprises a driving end 40, which is configured as a protrusion 60. In this example, the protrusion has a smaller cross section than the cross section of the suture attachment portion 39 (i.e. the central portion of the shuttle). The shuttle driver 13 comprises a tubular channel 61, which is sized and shaped to engage over the protrusion 60.

While the invention has been illustrated and described in detail in the drawings and foregoing description, such illustration and description are to be considered illustrative or exemplary and not restrictive, the invention being not limited to the disclosed embodiment. Other embodiments and variants are understood, and can be achieved by those skilled in the art when carrying out the claimed invention, based on a study of the drawings, the disclosure and the appended claims. New embodiments or variants may be obtained by combining any of the above teachings.

The above-described suturing device is a manually powered and operated for serving a suturing purpose. It should be noted that alternative actuation mechanisms and methods can achieve the same purpose. These actuation mechanisms do not need to be actuated by manually applied forces, but they may instead be motor-driven or magnet-driven mechanisms. For example, the shuttle driver 13 could be arranged over a motorised spindle, which actuates the rotational movement. Equally, the stack of suture holder assemblies may be actuated to translate by means of a motor. An example translation mechanism may be a conveyor belt. Furthermore, the described example suturing device comprises a shuttle driving mechanism that is configured to rotate about an axis. Alternatively, translational movements could pass the shuttle across the soft tissue clearance and achieve the same result.

In the claims, the word “comprising” or “including” does not exclude other elements or steps, and the indefinite article “a” or “an” does not exclude a plurality. The mere fact that different features are recited in mutually different dependent claims does not indicate that a combination of these features cannot be advantageously used. Any reference signs in the claims should not be construed as limiting the scope of the invention.

What is claimed is:

1. A surgical suturing device for closing an incision or two layers of a target soft tissue, the suturing device comprising:
a shuttle driving mechanism for driving a shuttle of a suture holder assembly; and
a chamber for receiving a set of suture holder assemblies,
the suturing device defining:
a soft tissue window for receiving at least a portion of the target tissue; and
an ejection window sized and shaped to allow a suture holder assembly to be ejected out of the suturing device through the ejection window,
wherein the suturing device defines at least an engaging state, and a transferring state, and wherein in the engaging state, the shuttle driving mechanism is in a first retracted position while being configured to be engaged with the respective shuttle of the respective suture holder assembly in the chamber, and in the transferring state, the shuttle driving mechanism is in a first advanced position such that at least a portion of the shuttle driving mechanism spans at least partially across the soft tissue window while being configured to be engaged with the respective shuttle of the respective suture holder assembly.

2. The suturing device according to claim 1, wherein the suturing device further defines a start state, in which the shuttle driving mechanism is in a second, different retracted position configured to be disengaged from the respective shuttle of the respective suture holder assembly.

3. The suturing device according to claim 1, wherein the suturing device further defines a tensioning position, wherein the shuttle driving mechanism is in a second advanced state such that at least a portion of the shuttle driving mechanism spans across the soft tissue window, and the shuttle driving mechanism is configured to be engaged with the respective shuttle of the respective suture holder assembly to allow a respective suture strand of the respective suture holder assembly to form a substantially slack-free loop around the target tissue in the soft tissue window.

4. The suturing device according to claim 1, wherein the suturing device comprises a feed mechanism in the chamber configured to feed the set of suture holder assemblies towards the ejection window, wherein the feed mechanism comprises a first biasing element, and wherein the first biasing element is configured as a first spring.

5. The suturing device according to claim 1, wherein the shuttle driving mechanism comprises a needle.

6. The suturing device according to claim 5, wherein the needle is curved and has a substantially constant radius.

7. The suturing device according to claim 5, wherein the needle comprises a shuttle engaging tip with a first cross-sectional diameter and a main body portion with a second cross-sectional diameter, wherein the second diameter is greater than the first diameter, and wherein the shuttle engaging tip is sized and shaped to be received in a shuttle channel in the respective shuttle of the respective suture holder assembly.

8. The suturing device according to claim 5, wherein the shuttle comprises a driving end configured as a protrusion, wherein a shuttle driver of the shuttle driving mechanism comprises a shuttle driver channel, which is sized and shaped to receive the protrusion.

9. The suturing device according to claim 5, wherein the shuttle driving mechanism comprises an arm coupled to the needle, and wherein the arm is arranged to rotate about a first axis.

10. The suturing device according to claim 9, wherein the arm comprises a first tooth section, and wherein the first tooth section is configured as a first cogwheel section.

11. The suturing device according to claim 5, wherein the suturing device comprises a groove or device channel, and wherein the needle is received in the groove or device channel.

12. The suturing device according to claim 1, wherein the suturing device comprises an activation mechanism coupled to the shuttle driving mechanism, and wherein the activation mechanism comprises a lever.

13. The suturing device according to claim 12, wherein the activation mechanism comprises a second tooth section for engaging with the shuttle driving mechanism, wherein the second tooth section is configured as a second cogwheel section, and wherein the second tooth section is configured to engage with a first tooth section of the shuttle driving mechanism.

14. The suturing device according to claim 12, wherein the activation mechanism is configured to rotate about a second axis.

15. The suturing device according to claim 12, wherein the suturing device comprises a second biasing element configured to urge the activation mechanism, and wherein the second biasing element is configured as a second spring.

16. The suturing device according to claim 1, wherein the chamber is delimited by a set of wall sections, and wherein at least one of the wall sections comprises a stop element configured to engage against the respective shuttle of the respective suture holder assembly to prevent inadvertent ejection of the respective suture holder assembly out of the chamber.

17. A surgical suturing tool assembly for closing an incision or two layers of a target soft tissue, the suturing tool assembly comprising the suturing device according to claim 1, and a set of suture holder assemblies, the respective suture holder assembly comprising a shuttle, a suture strand attached to the shuttle, and a holder with a first holder end and a second holder end and a soft tissue clearance between the first and second holder ends, wherein the respective suture strand comprises a first suture strand end attached to the shuttle, and a second suture strand end.

18. The suturing tool assembly according to claim 17, wherein the shuttle is an elongated tubular element.

19. The suturing tool assembly according to claim 17, wherein in the engaging state, the respective shuttle is arranged at the first holder end and engaged with the shuttle driving mechanism, and in the transferring state, the respective shuttle is a arranged at the second holder end and engaged with the shuttle driving mechanism to allow the respective suture strand of the respective suture holder assembly to form a loop around the target tissue in the soft tissue window.

20. The suturing tool assembly according to claim 17, wherein the respective suture holder assembly comprises a blade for cutting off the respective suture strand.

21. The suturing tool assembly according to claim 17, wherein the respective suture holder assembly comprises a shuttle receiving channel at the second holder end and is configured to receive the respective shuttle, and wherein the shuttle receiving channel is further configured to receive at least a portion of the shuttle driving mechanism.

22. The suturing tool assembly according to claim 17, wherein the respective suture holder assembly comprises a suture retention element for receiving the respective suture strand, and wherein the suture retention element is configured to allow the respective shuttle to pass through the suture retention element.

23. A suture holder assembly comprising a shuttle, a suture strand attached to the shuttle, and a holder with a first holder end, a second holder end and a soft tissue clearance between the first and second holder ends, wherein the respective suture strand comprises a first suture strand end attached to the shuttle, and a second suture strand end, and wherein the shuttle is configured to travel between the first and second holder ends when suturing a target tissue.

24. The suture holder assembly according to claim 23, wherein the suture holder assembly further comprises a blade for cutting off the suture strand.

25. The suture holder assembly according to claim 23, wherein the suture holder assembly further comprises a shuttle receiving channel at the second holder end and is configured to receive the shuttle.

26. The suture holder assembly according to claim 23, wherein the suture holder assembly further comprises a suture retention element for holding the second suture strand end, and wherein the suture retention element is configured to allow the shuttle to pass through the suture retention element.
